(19) United States
(12) Patent Application Publication
(10) Pub. No.: US 2022/0080469 A1
(43) Pub. Date: Mar. 17, 2022

(54) MEDICAL DEVICE CLEANING DEVICES AND METHODS

(71) Applicant: CLARUS MEDICAL, LLC, Minneapolis, MN (US)

(72) Inventors: Scott Allen SUNDET, Edina, MN (US); Cindy Trosen SUNDET, Edina, MN (US); Randal Alan GATZKE, Minneapolis, MN (US); Mark BROWN, Minneapolis, MN (US); Thomas Chandler, Coon Rapids, MN (US); Kristin Sundet PAVEK, Golden Valley, MN (US)

(21) Appl. No.: 17/471,578

(22) Filed: Sep. 10, 2021

Related U.S. Application Data
(60) Provisional application No. 63/077,025, filed on Sep. 11, 2020.

(51) Int. Cl. B08B 7/00; A61B 90/70; B08B 9/043; G02B 23/24; A61L 2/10

(52) U.S. Cl. CPC B08B 7/0057; A61B 90/70; B08B 9/043; G02B 23/2423; A61L 2202/24; A61L 2/10; A61B 2090/701; B08B 2209/04; G02B 23/2484

(57) ABSTRACT

A method for inspecting and cleaning an inner surface of a medical device may involve advancing a distal end of a flexible, elongate inspection device into the medical device, visualizing the inner surface of the medical device with a visualization member at or near the distal end of the inspection device, and emitting an ultraviolet light from an ultraviolet light emitter at or near the distal end of the inspection device, to clean the inner surface. The ultraviolet light emitter is configured to emit a uniform beam of ultraviolet light onto the inner surface.

### CROSS-REFERENCE TO RELATED APPLICATIONS

This application claims the benefit of U.S. Provisional Patent Application No. 63/077,025, filed Sep. 11, 2020, entitled, “MEDICAL DEVICE CLEANING DEVICES AND METHODS.” The disclosure of this priority application is hereby incorporated by reference in its entirety into the present application.

### FIELD

This application is directed to medical devices, systems and methods. More specifically, the application is directed to devices, systems and methods for inspecting and/or cleaning medical devices.

### BACKGROUND

Millions of medical devices are used in hospitals throughout the world every day. With the continuing advancement of medical and surgical procedures over time, one of the trends for many years is toward minimally invasive procedures performed through smaller incisions or even through the body's natural orifices. Examples of this trend include arthroscopic surgery, transcatheter aortic valve replacement (“TAVR”), natural orifice transluminal endoscopic surgery (“NOTES”), robotic surgery and many others. Many of these procedures involve the use of long, flexible catheter instruments, long, thin, rigid, instruments with lumens, and/or long, flexible endoscopes for visualizing the procedure. Additionally, endoscopes are used in countless different diagnostic and therapeutic procedures in many parts of the body.

One of the challenges with the use of endoscopes, fiber scopes, catheter-based medical/surgical instruments and other long, thin, reusable instruments is how to properly and effectively clean them, especially their inner lumens. Many endoscopes and other instruments are too expensive to be disposable and so must be reused. And long, small-diameter, flexible instruments can be extremely hard to clean on the inside. They are also hard to inspect on the inside. Not only can flexible instruments collect bacteria and other contaminants, but they can also crack or become otherwise permanently deformed during use, for example when the instrument is bent or kinked. These instruments are typically processed in a cleaning facility located within the hospital, by workers with very little training. To inspect the inside of such instruments, a small, flexible scope is inserted and advanced through the lumen(s) of the device, so that contaminants and damage can be seen. It can be difficult, however, for the person doing the inspection to effectively identify contaminants and internal damage to the device. Thus, the inspection process can be labor intensive and sometimes ineffective. It can also be hard to find a scope small enough to fit through the lumens of some medical devices while allowing for adequate visualization. Additionally, once contamination of an endoscope or catheter lumen (or similar inner portion of a medical device) is identified, it can often be difficult to adequately clean and/or decontaminate the lumen.

Therefore, it would be desirable to have improved devices, systems and methods for inspecting medical devices, specifically endoscopes, catheters and other long, thin (flexible or rigid) medical devices with inner lumens, which are difficult to inspect on the inside. It would also be advantageous to have devices, systems and methods for cleaning/decontaminating/disinfecting the inner portions of such devices. At least some of these objectives are addressed in this application.

### SUMMARY

According to one aspect of the present disclosure, a medical device inspection system includes: a base; a medical device holder on the base, for holding a medical device to be inspected; a fiber scope holder, for holding a handle of a fiber scope; a moveable roller moveably attached to the base such that it is free to rotate around an axis and move from a first position to a second position along the base; and a feeder coupled with the base for feeding a flexible portion of the fiber scope into a lumen of the medical device. During use, the flexible portion of the fiber scope extends from the handle, around the moveable roller, and through the feeder to enter an opening in the lumen of the medical device. The moveable roller is configured to move from the first position that is farthest away from the handle and the feeder to the second position that is closer to the handle and the feeder as the flexible portion is fed into the lumen of the medical device.

In various embodiments, the medical device holder may be a clamp or similar holding device. The feeder may sometimes include a first spinning drum, a drive mechanism attached to the first spinning drum, and a second spinning drum. The flexible portion of the fiber scope may pass between, and be advanced by, the first and second spinning drums. In some embodiments, a contact surface of the first spinning drum and/or a contact surface of the second spinning drum may be made at least partially of a compliant polymer. Some embodiments may also include a tensioner coupled with the feeder to adjust an amount of force applied between the first and second spinning drums.

Some embodiments may also optionally include a controller attached to the feeder, where the controller includes a processor with computer readable instructions for causing the feeder to advance the fiber scope automatically into the lumen of the medical device. In some embodiments, the controller causes the feeder to advance the fiber scope automatically into the lumen in incremental and measurable steps. Optionally, the controller may be further in communication with the fiber scope, and the processor may be configured to instruct the fiber scope to capture images at multiple positions within the lumen of the medical device. In some embodiments, the processor is further configured to determine, from an image captured by the fiber scope, that the lumen contains a defect, and instruct the feeder and/or the fiber scope to record a location of the defect in the lumen. For example, the location may be a distance from the opening in the lumen to the defect. In some embodiments, the processor may use artificial intelligence to determine that the lumen contains the defect. For example, the processor may use artificial intelligence to distinguish differently labeled shapes within the lumen of the medical device, such as normal, gouged, oval, wet and debris-containing. In various embodiments, the processor may use artificial intelligence to record an image, a location, a description, a date, a time, a name of a person operating the system, and/or a recommended course of corrective action pertaining to an identified defect in the lumen of the medical device. In some embodiments, artificial intelligence is embodied in an artificial intelligence chip located the base, the feeder, or the fiber scope.

In some embodiments, the handle of the fiber scope and the medical device are attached to the base, such that they face in the same direction, toward the moveable roller. Some embodiments of the system may further include a first fixed roller fixedly attached to the base between the moveable roller and the feeder, where the handle of the fiber scope and the medical device face in different directions. In some embodiments, the system may include a first fixed roller fixedly attached to the base between the moveable roller and the feeder and a second fixed roller fixedly attached to the base between the moveable roller and the fiber scope. In such embodiments, the handle of the fiber scope and the medical device may face toward one another.

In some embodiments, the system may include the fiber scope. For example, the fiber scope may be a water resistant fiber scope. The scope may include multiple internal applications of adhesive to provide water resistance. Optionally, the fiber scope may include an ultraviolet light emitter for emitting light onto a contaminated portion, or entirety, of the lumen of the medical device to help treat the contaminated portion. The fiber scope may further include a light diffuser at or near a distal tip of the fiber scope for diffusing the emitted ultraviolet light. The diffuser is used to provide a 360 degree radial delivery of the entirety of the circumference of the internal lumen. The fiber scope may further include a handle and a flexible portion. The flexible portion may include a sheath, a laser fiber disposed in the sheath to provide kink resistance, at least one light emitting fiber, and at least one image capturing fiber. In alternative embodiments, the fiber scope may include an image capturing chip at a distal end of the sheath. The scope may also include a lock-out feature that prevents use of the fiber scope after a predetermined number of uses. The feeder may include a torque sensor to prevent applying excessive force to the flexible portion of the fiber scope.

In another aspect of the present disclosure, a medical device inspection system may include: a base; a medical device holder on the base, for holding a medical device to be inspected; a roller attached to the base such that it is free to rotate around an axis, wherein the roller holds a flexible portion of a fiber scope; a feeder coupled with the base for feeding the flexible portion of the fiber scope from the roller into a lumen of the medical device; and a communications module for transmitting images captured by the flexible portion of the fiber scope to a handle or other control portion of the fiber scope. During use, the flexible portion of the fiber scope extends from the roller through the feeder to enter an opening in the lumen of the medical device. The communications module may, for example, be a Bluetooth communication module.

In another aspect of the present disclosure, a method for inspecting an inside of a medical device may involve: positioning a flexible fiber scope around a portion of a first roller of a medical device inspection system, such that a handle of the flexible fiber scope is positioned on one side of the first roller and a feeder of the medical device inspection system is on an opposite side of the roller; positioning the flexible fiber scope through the feeder; advancing a distal end of the flexible fiber scope into an opening in a lumen of the medical device; advancing the distal end of the flexible fiber scope farther into the lumen of the medical device, using the feeder, where advancing the distal end farther causes the first roller to turn around an axis and move along the medical device inspection system toward the handle and the feeder; and capturing at least one image of the lumen of the medical device with the flexible fiber scope.

In some embodiments, the method may further involve attaching the medical device to the medical device inspection system before advancing the distal end of the fiber scope into the lumen. In some embodiments, positioning the flexible fiber scope through the feeder may involve positioning the flexible fiber scope between a first spinning drum and a second spinning drum of the feeder. Embodiments may also involve adjusting a tensioner of the medical device inspection system to adjust an amount of force applied to the flexible fiber scope by the first and second spinning drums. In some embodiments, advancing the distal end of the flexible fiber scope farther into the lumen of the medical device is performed automatically by the feeder in a stepwise fashion. Some embodiments may also include recording, with the medical device inspection system, multiple distances into the lumen of the medical device at which images are captured by the flexible fiber scope.

In some embodiments, a controller of the medical device inspection system instructs the flexible fiber scope to acquire at least one image. Optionally, the method may also involve determining, from an image captured by the fiber scope, that the lumen contains a defect, and instructing the feeder and/or the fiber scope to record a location of the defect in the lumen. Some embodiments may further involve determining a distance from the opening in the lumen to a defect in the lumen, using a processor of the medical device inspection system. The method may also involve using artificial intelligence in the medical device inspection system to determine that the lumen contains the defect. Optionally, the method may also involve using the artificial intelligence to distinguish differently labeled shapes within the lumen of the medical device. The method may also involve using the artificial intelligence to record an image, a location, a description, a date, a time, a name of a person operating the system, and/or a recommended course of corrective action pertaining to an identified defect in the lumen of the medical device.

In some embodiments, the method may also involve attaching the handle of the fiber scope to the medical device inspection system. The method may also involve positioning the flexible fiber scope around a first fixed roller fixedly attached to the medical device inspection system between the roller and the feeder. The method may also involve positioning the flexible fiber scope around a second fixed roller fixedly attached to the medical device inspection system between the roller and the handle of the flexible fiber scope. Optionally, the method may involve emitting ultraviolet light from the flexible fiber scope onto a contaminated portion of the lumen of the medical device to help treat the contaminated portion. Such an embodiment may further involve diffusing the ultraviolet light with a light diffuser before emitting it from the flexible fiber scope. In some embodiments, the ultraviolet light may be emitting pulsed light.

In some embodiments, the method may also involve preventing kinking of the flexible fiber scope by housing a laser fiber in a sheath of the flexible fiber scope. In some embodiments, the method may also involve preventing the flexible fiber scope from being used more than a predetermined number of times by including a lock-out feature in the medical device inspection system. The method may further involve preventing the flexible fiber scope from being used with unapproved medical devices or by unapproved inspection personnel by including a lock-out feature in the medical device inspection system. The method may also involve sensing an amount of torque applied to the flexible fiber scope by the medical device inspection system to prevent applying excessive force to the flexible fiber scope.

Another aspect of the present disclosure includes a method for disinfecting an inner surface of a channel of an endoscope, catheter or other medical instrument having a channel. The method includes advancing a laser fiber assembly through the channel; the laser fiber assembly includes an optical fiber having a radial light diffuser. The method further includes transmitting a UV light from a light source through the optical fiber at a predetermined power level. The transmission of the UV light causes emission of the UV light from the radial diffuser in a radial illumination pattern on the inner surface of the channel serving to irradiate and disinfect the inner surface.

These and other aspects and embodiments are described more fully below, in reference to the attached drawing figures.

### BRIEF DESCRIPTION OF THE DRAWINGS

FIG. 1 is a diagrammatic representation of one example of a medical device inspection system;

FIG. 2 is a diagrammatic representation of another example of a medical device inspection system;

FIG. 3 is a diagrammatic representation of another example of a medical device inspection system;

FIG. 4 is a diagrammatic representation of another example of a medical device inspection system;

FIG. 5 is a diagrammatic representation of example shapes of an interior of a lumen of a medical endoscope, illustrating a method according to one example;

FIG. 6 is a flow diagram of a method of inspecting a medical device, according to one example;

FIG. 7 is a perspective view of a medical device inspection scope, according to one embodiment;

FIG. 8 is a cross-sectional view of the inspection scope of FIG. 7; and

FIG. 9 is a perspective view of a medical device inspection and decontamination scope, according to another embodiment.

### DETAILED DESCRIPTION

Disclosed in this application are various examples of a medical device inspection system and method. In general, the system and method provide for automatic feeding of a fiber scope into the lumen of a medical device in order to inspect the medical device. Feeding of the fiber scope may be done in a stepwise fashion, images may be captured at specified intervals, and the locations of the intervals may be recorded. In some embodiments, artificial intelligence may be used to help the operator of the system identify imperfections in the lumen of the medical device, such as contaminations and defects. These concepts and many others are described in greater detail below. The examples described herein are not intended to limit the scope of the invention but are provided for descriptive purposes only.

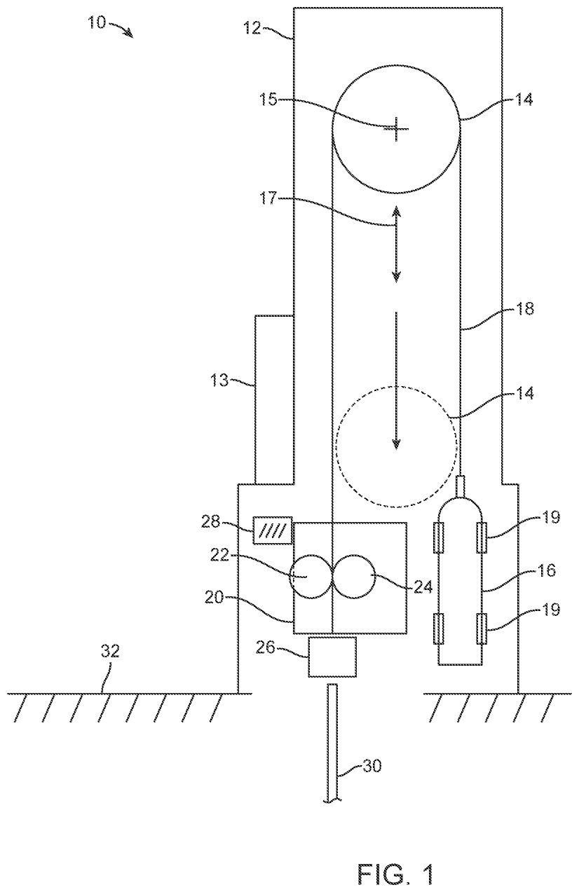

Referring now to FIG. 1, in one example, a medical device inspection and/or cleaning system 10 may include a base 12 (or “tower,” in the case of a vertical arrangement, as shown in FIG. 1) and multiple components attached to or embedded in base 12. (In this disclosure, the various described embodiments of a medical device inspection and/or cleaning system may be referred to simply as a “system,” for brevity.) This embodiment of system 10 is shown with the bottom of base 12 resting on a flat surface 32, such as a table. In another example, inspection system 10 may be flipped, so that base 12 hangs from a ceiling or other elevated structure. In yet another example, base 12 may lie flat on flat surface 32. Attached to base 12 is a moveable roller 14, which is free to rotate about its central axis of rotation 15 and also move back and forth along a longitudinal axis 17 of base 12. Also attached to base 12 is a fiber scope handle attachment member 19, by which a fiber scope handle 16 is attached to base 12. Handle attachment member 19 may be multiple clips (as shown), clamps or any other suitable attachment structure(s).

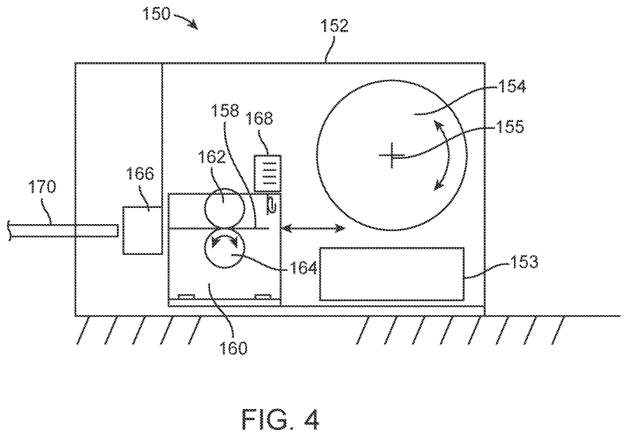

Regarding the fiber scope shown in FIG. 1 and subsequent figures, the term “fiber scope” is used in this application to mean any type of elongate, flexible scope device, including a fiber optic scope and/or a digital scope. The fiber scope is shown as including a handle 16, which may include a processor and a light source for the scope, and a flexible, image capturing portion 18, which passes around moveable roller 14 to a feeder 20, which is also attached to base 12. Image capturing portion 18 may include a sheath, one or more fiber optic fibers, a “camera on a chip,” such as a CMOS camera, and/or the like. In some embodiments (for example, the one shown in FIG. 4), the scope may not include a handle but may house the light source and processor elsewhere.

Feeder 20 includes a first spinning drum 22 and a second spinning drum 24. One of the two drums 22, 24, in this example first drum 22, is connected to a drive mechanism or motor (not shown), which spins the first drum 22 about its axis. Second drum 24 spins freely when pressure from first drum 22 is applied to it and the motor spins first drum 22. Feeder 20 also includes a tensioner 28 for adjusting an amount of tension between the two surfaces of the two drums 22, 24 against one another. System 10 also includes a medical device attachment clamp 26 (or other attachment member) coupled with base 12, for attaching medical device 30 to system 10. Neither medical device 30 nor surface 32 is typically part of system 10, but they are illustrated for exemplary purposes. Finally, system 10 may include a display and control module 13 (or multiple modules). Display and control module 13 may include a display portion, such as a video monitor, with or without touch screen capabilities. Module 13 may also include one or more controls for controlling feeder 20, controlling the fiber scope and the like. The display portion of module 13 may show images taken with the fiber scope, indicator light(s) signifying a contaminated or damaged area in the lumen of medical device 30, information about a contamination or damaged area in medical device 30, identifying information identifying medical device 30 and/or any other suitable information.

In use, handle 16 is attached to handle attachment member 19, and flexible scope portion 18 is passed around moveable roller and through drums 22, 24. Tensioner 28 is adjusted to adjust the tension placed on flexible portion 18 by drums 22, 24. In one embodiment, the contact (outer) surfaces of drums 22, 24 may be made of nylon or other polymer and may be somewhat compliant, to better advance flexible portion 18 without damaging it. Medical device 30 is attached to clamp 26, and the distal end of flexible portion 18 is advanced into an opening in a lumen at the distal end of medical device 30. Feeder 20 then feeds flexible portion 18 of the fiber scope farther and farther into the lumen of medical device, until the entire device 30 (or a desired portion of device 30) is inspected. As fiber scope 18 advances, it may take multiple still images and/or video images of the lumen. As more and more of fiber scope 18 is advanced into medical device 30, moveable roller 14 moves longitudinally (or “translates”) along base 12 from a first position (solid-lined version at the top of FIG. 1) to a second position (dotted-line version at the bottom of FIG. 1), to accommodate for the shorter amount of flexible portion 18 between handle 16 and medical device 30. At the same time, moveable roller 14 also rotates around its rotational axis 15. As illustrated, in the second position, roller 14 is closer to handle 16 and feeder 20 than in the first position. Once flexible portion 18 has been advanced as far into medical device 30 as desired, it is retracted by handle 16, roller 14 and feeder 20, and roller 14 moves back to the first position from the second position.

Control and display module 13 may include any type of computing device, including a processor, and the processor may contain instructions for driving feeder 20 and/or the fiber scope. For example, in some embodiments, the drive mechanism of feeder 20 may be a step motor, and the controller may control incremental advancement of flexible portion 18 into medical device 30 via feeder 20. In some examples, the processor may include an artificial intelligence chip or other mechanism for artificial intelligence. Artificial intelligence may be used, for example, to allow the processor to identify irregularities inside the lumen of medical device 30 in images of the lumen captured by the fiber scope. For example, the processor may be able to identify contaminants, biofilms, gouges, kinks, cracks, moisture and/or the like inside medical device 30. This identification may be enhanced via artificial intelligence, where the processor has been “taught” to detect irregularities by learning images of similar irregularities in other medical devices. In some embodiments, the processor may be used to detect an irregularity in the lumen during advancement, instruct the fiber scope to capture an image of the irregularity, determine a location of the irregularity in the form of a distance of the irregularity from a distal opening of the lumen, and store identifying information about the type and location of the irregularity in the controller. The controller may also store additional information, such as the type of medical device 30 being examined, the date, the time, the identity of the personnel conducting the examination, how many times the particular fiber scope has been used to inspect medical devices, and/or the like. In some embodiments, feeder 20 feeds flexible portion 18 into medical device 30 in predetermined increments, and an image is taken by the fiber scope at each increment. In other embodiments, the fiber scope may take continual video footage throughout the advancement, and the scope may also take still images at any identified areas of irregularity. Virtually any combination of fiber scope advancement and image capture is possible, and the controller/processor of system 10 may be capable of controlling any of a large number of different protocols.

In some embodiments, system 10 may also be capable of cleaning, decontaminating and/or disinfecting the inside (lumen) of medical device 30. For purposes of this application, the term “cleaning” is meant to include any type of cleaning, decontaminating, disinfecting, sterilizing or the like. For example, system 10 may be designed to emit ultraviolet (UV) light at one or more wavelength, such as but not limited to UVC light, to decontaminate an inner lumen of medical device 30. In some embodiments, system 10 may use UV visualization to detect a contaminant, such as but not limited to a biofilm, in the inner lumen of medical device 30, and then may also use UV light, such as UVC light, to decontaminate the inner lumen. When using UVC light to decontaminate a medical device, delivering the UVC light in a uniform manner may be critical to the success of the decontamination. System 10 may achieve this uniform delivery by automatically advancing the fiber scope at a uniform speed, by including a diffuser or similar structure at the distal end of the fiber scope to diffuse the emitted UVC light, or a combination of both. In some embodiments, both the inspection and the cleaning functions may be performed automatically by system 10, thus negating the need for manual human control, reducing the incidence of human error, and reducing the need for training of employees. In some embodiments, artificial intelligence (AI) may be used by system 10 for detection of a biofilm and/or other contaminants and for the cleaning/decontamination function. For example, AI may identify a contaminant and send a signal to the processor of system 10 to turn on UVC light to automatically begin decontamination. Or system 10 may identify biofilms and other contaminants in a medical device lumen, remember the identities and locations of the contaminants, and then, after the identifying step, system 10 may tell the cleaning module of system 10 how to clean the inside of medical device 30, based on the remembered information.

System 10 may be used to inspect and/or clean any suitable medical or surgical device. The types of devices may include endoscopes of any kind, catheters, flexible or rigid instruments with lumens or channels, or virtually any other type of device with channel having an internal surface that is hard to inspect visually from outside the device. In certain embodiments, the channel has a diameter of less than one inch. In certain embodiments, the channel has a diameter of less than a half-inch. In certain embodiments the channel has a diameter of less than a quarter-inch. In certain embodiments, the channel has a diameter of less than an eighth-inch. Other channel diameters are also possible. System 10 may also have a number of different sizes and shapes in different examples. In the example of FIG. 1, when roller 14 moves all the way to the second position (dotted lines), it may be necessary to have about 12 inches of extra flexible portion 18 of the fiber scope, in order to extend from the distal end of handle 16, around roller 14, through feeder 20 and into the distal end of medical device 30. Other examples of system 10 may require shorter or longer segments of flexible portion.

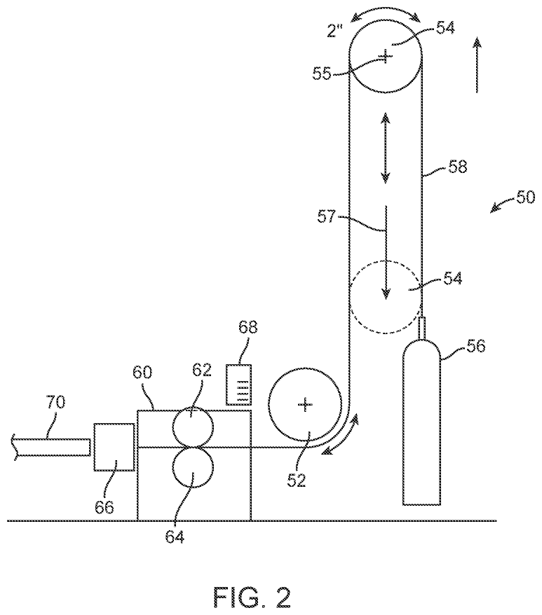

Referring now to FIG. 2, another example of a medical device inspection and/or cleaning system 50 is illustrated diagrammatically. FIG. 2 does not illustrate a base, tower or platform of system 50, and it also does not show the control/display module, but these features may be the same as, or similar to, those shown in FIG. 1, with the exception that the overall system 50 is L-shaped. Similar to the example of FIG. 1, system 50 includes a moveable roller 54, which is rotatable about its own axis 55 and also slidable along a longitudinal axis 57, from a first position to a second position (and back). Roller 54 may be spring loaded (same as in the previous example), so that when it is not under tension it returns to the first position. System 50 also includes a fiber scope with a handle 56 and a flexible portion 58, as well as a feeder 60 with a first spinning drum 62, a second spinning drum 64, a tensioner 68 and a medical device clamp 66, all as described above. A medical device 70 is typically not part of system 50, but rather is the item inspected by system 50.

The main difference between system 50 of FIG. 2 and system 10 of FIG. 1 is that the former includes a second, fixed-location roller 52, about which flexible portion 58 of the fiber scope wraps. This allows the two rollers 52, 54 to act as two pulleys, which may enhance feeding of flexible portion into medical device 70. This example may require a longer extra portion of flexible portion than in the previous example, such as about 14 inches to about 16 inches in one example. In all other respects, system 50 may share any or all of the characteristics and features of system 10 described above.

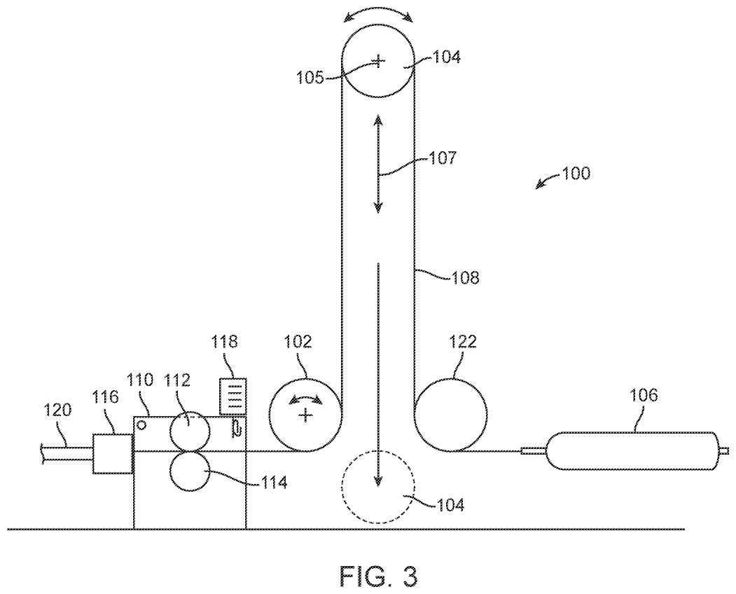

Referring now to FIG. 3, another example of a medical device inspection and/or cleaning system 100 is illustrated diagrammatically. FIG. 3 also does not illustrate a base, platform or tower for holding system 100, but it may be similar to that shown in FIG. 1, with the exception that the overall system 100 has a three-prong (or triangular) shape. Similar to the example of FIG. 2, system 100 includes a moveable roller 104, which is rotatable about its own axis 105 and also slidable along a longitudinal axis 107 from a first position to a second position (and back). Roller 104 may be spring loaded, as described previously. System 100 also includes a fiber scope with a handle 106 and a flexible portion 108, as well as a feeder 110 with a first spinning drum 112, a second spinning drum 114, a tensioner 118 and a medical device clamp 116, all as described above. A medical device 120 is typically not part of system 100, but rather is the item inspected by system 100.

Like system 50 of FIG. 2, system 100 of FIG. 3 includes a second, fixed-location roller 102, about which flexible portion 108 of the fiber scope wraps. Additionally, system 100 includes a fixed cylinder 122, about which flexible portion 108 also wraps. In this example, fixed cylinder 122 does not rotate but merely acts as a third surface about which flexible portion 108 curves, thus providing system 100 with effectively three pulleys. This example may require about 12 inches of extra flexible portion 108 in one example. In all other respects, system 100 may share any or all of the characteristics and features of systems 10 and 50 described above.

Referring now to FIG. 4, another example of a medical device inspection and/or cleaning system 150 is illustrated diagrammatically. This example of system 150 includes a base 152 and one large roller 154, which rotates about its own axis 155 but is fixed relative to base 152 (e.g., it does not move laterally along base 152). In this embodiment, the fiber scope is housed within roller 154. The processor and light source that are housed in a handle in other embodiments are housed somewhere within roller 154, and the flexible portion 158 is also housed in roller 154 in a spool-like fashion. To advance flexible portion 158 of the fiber scope into medical device 170, roller 154 rotates to unspool flexible portion 158. As in previous embodiments, system 150 also includes a display and control module 153 and a feeder 160 with a first spinning drum 162, a second spinning drum 164, a tensioner 168 and a medical device clamp 166, all as described above. A medical device 170 is typically not part of system 150, but rather is the item inspected by system 150.

The main difference between system 150 of FIG. 4 and previously described embodiments is roller 154, and the housing of the fiber scope in roller 154. In all other respects, system 150 may share any or all of the characteristics and features the previously described embodiments.

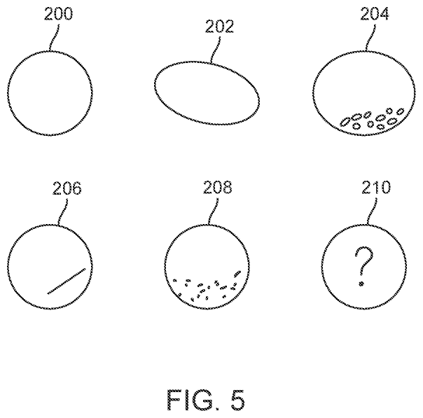

Referring now to FIG. 5, as mentioned briefly above, in some examples the medical device inspection system may include a computer processor with artificial intelligence (AI) capabilities and/or instructions for running an algorithm, either or both of which may allow the system to identify abnormalities within a medical device and even label the abnormalities according to types. For example, the system processor may be able to learn multiple shapes of medical device lumens and identify abnormalities by shape. FIG. 5 illustrates just several examples of such learned shapes. These examples include normal 200, kinked 202 (or oval), wet 204 (signifying accumulated moisture in the device), gouged 206, debris 208 (which could include contamination) and unidentified 210. The last of these-unidentified 210-could be a shape or collection of shapes that the system processor is able to identify as abnormal but is not able to assign to a particular category.

In various examples, the system may do any or all of the following. (1) The feeder may advance the camera through the medical device lumen in stepwise fashion or continuously until the system identifies an abnormality in the lumen, at which point the system may automatically stop advancing the camera and capture a video or still image of the area with the abnormality. (2) The system may identify the abnormality in the lumen based on learned shapes of images of medical device lumens stored in the system's processor. (3) The system may display the irregularity on the system display with some kind of label, such as a word description and/or an indicator light. (4) The system may provide other information about the irregularity, such as its location in the lumen (a distance from one end of the medical device, for example). (5) The system may automatically emit a UV light (such as but not limited to UVC light) to disinfect an identified contamination in the lumen. Any combination of these activities, as well as others, may be performed by the system.

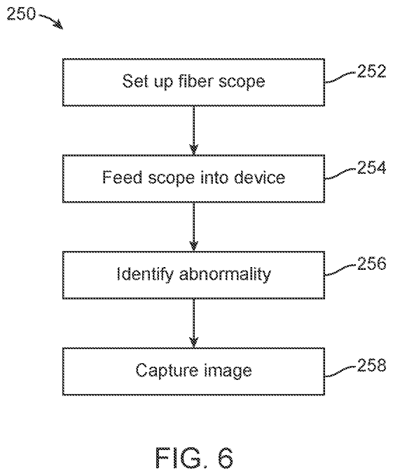

Referring now to FIG. 6, one example of a method 250 for inspecting a medical device, such as a lumen (or “channel”) of an endoscope is described. According to one example, a method 250 for inspecting an inside of a medical device may first involve setting up 252 or positioning the fiber scope on or in the medical device inspection system. This step of setting up 252 may include, for example, positioning a flexible fiber scope around a portion of a first roller of a medical device inspection system, such that a handle of the flexible fiber scope is positioned on one side of the first roller and a feeder of the medical device inspection system is on an opposite side of the roller. The method 250 may then involve positioning the flexible fiber scope through the feeder and advancing 254 a distal end of the flexible fiber scope into an opening in a lumen of the medical device. Next, the distal end of the flexible fiber scope is advanced farther into the lumen of the medical device, using the feeder. In some embodiments, all advancement 254 of the fiber scope into the medical device is done through the feeder. Alternatively, the initial advancement 254 may be performed manually, and subsequent advancement 254 may be with the feeder. This advancement 254, in some examples, causes the first roller to turn around an axis and move along the medical device inspection system toward the handle and the feeder. In other embodiments, the first roller does not move along the medical device.

The method 250 may further include identifying an abnormality 256 in the medical device lumen. This identification step may be achieved using the processor, and in some cases artificial intelligence, of the system. Finally, the method 250 may include capturing at least one image 258 of the lumen of the medical device with the flexible fiber scope. This image capturing step 258 may be done automatically in some examples, where the processor identifies the abnormality and sends a signal to the camera to capture the image 258.

In some examples, the method 250 also includes attaching the medical device to the medical device inspection system before advancing the distal end of the fiber scope into the lumen. In some embodiments, positioning the flexible fiber scope through the feeder may involve positioning the flexible fiber scope between a first spinning drum and a second spinning drum of the feeder. Some embodiments may further include adjusting a tensioner of the medical device inspection system to adjust an amount of force applied to the flexible fiber scope by the first and second spinning drums. Advancing the distal end of the flexible fiber scope farther into the lumen of the medical device may sometimes be performed automatically by the feeder in a stepwise fashion.

Some examples of the method 250 may include recording, with the medical device inspection system, multiple distances into the lumen of the medical device at which images are captured by the flexible fiber scope. In some examples, a controller (or processor) of the medical device inspection system instructs the flexible fiber scope to acquire at least one image. In some examples, the method 250 may include distinguishing, from an image captured by the fiber scope, that the lumen contains a defect, and instructing the feeder and/or the fiber scope to record a location of the defect in the lumen. Some examples may involve determining a distance from the opening in the lumen to a defect in the lumen, using a processor of the medical device inspection system.

Some examples of the method 250 involve using artificial intelligence in the medical device inspection system to determine that the lumen contains the defect. Some examples of the method 250 involve using the artificial intelligence to distinguish differently labeled shapes within the lumen of the medical device, where the differently labeled shapes may include normal, gouged, oval, wet and debris-containing. The artificial intelligence may also be used to record an image, a location, a description, a date, a time, a name of a person operating the system, and/or a recommended course of corrective action pertaining to an identified defect in the lumen of the medical device.

In some examples, the method 250 may involve attaching the handle of the fiber scope to the medical device inspection system. Some embodiments may involve positioning the flexible fiber scope around a first fixed roller fixedly attached to the medical device inspection system between the roller and the feeder. Some examples may involve positioning the flexible fiber scope around a second fixed roller fixedly attached to the medical device inspection system between the roller and the handle of the flexible fiber scope.

As discussed above, the method 250 may additionally include one or more cleaning/disinfecting steps. For example, in some embodiments, the fiber scope may be used to fully inspect the inside of the medical device, retracted out of the device, and then advanced back into the device to perform the cleaning function. In other embodiments, inspection and cleaning may be performed during one pass of the fiber scope into the medical device—e.g., the fiber scope identifies contaminants and decontaminates immediately. Any suitable cleaning methods and protocols may be used, according to various embodiments. For example, in some embodiments, the method 250 may involve emitting ultraviolet light from the flexible fiber scope onto a contaminated portion of the lumen of the medical device to help treat the contaminated portion. Alternatively, any other type or wavelength of light may be emitted to treat a contaminated area in a medical device. Light may alternatively or additionally be emitted to help identify an area of damage or contamination in the medical device lumen. For example, chemoluminescence may be used in some examples. In some examples, the method 250 may also include diffusing the ultraviolet light (or other form of light) with a light diffuser before emitting it from the flexible fiber scope. In some embodiments, emitting the ultraviolet light involves emitting pulsed light.

The method 250 may also involve preventing kinking of the flexible fiber scope by housing a laser fiber in a sheath of the flexible fiber scope. The method 250 may also involve preventing the flexible fiber scope from being used more than a predetermined number of times by including a lock-out feature in the medical device inspection system. The method 250 may also involve preventing the flexible fiber scope from being used with unapproved medical devices or by unapproved inspection personnel by including a lock-out feature in the medical device inspection system. The method 250 may also involve sensing an amount of torque applied to the flexible fiber scope by the medical device inspection system to prevent applying excessive force to the flexible fiber scope.

Also disclosed in this application are various examples of an endoscope (or “fiber scope” or simply “scope”), which may be used for inspecting and/or cleaning/decontaminating the inside of medical devices, or for any other suitable purpose. For example, the fiber scope may be inserted into a lumen of a larger endoscope and advanced through the lumen to detect imperfections, damage, biofilm, contamination and/or the like inside of the endoscope. The scope may then be used to clean the lumen of the device, for example by emitting UVC light, other wavelength(s) of UV light, or alternative cleaning substances. In this way, the scope described in this application may help a user inspect a medical device that is to be cleaned, sterilized or otherwise processed for reuse. In some embodiments, the scope may be designed not only to help visualize imperfections and contamination of a medical device lumen, but also to disinfect or otherwise clean the lumen. These concepts are described in greater detail below. The examples of various features and embodiments of the fiber scope described below are not intended to limit the scope of the invention but are provided for descriptive purposes only.

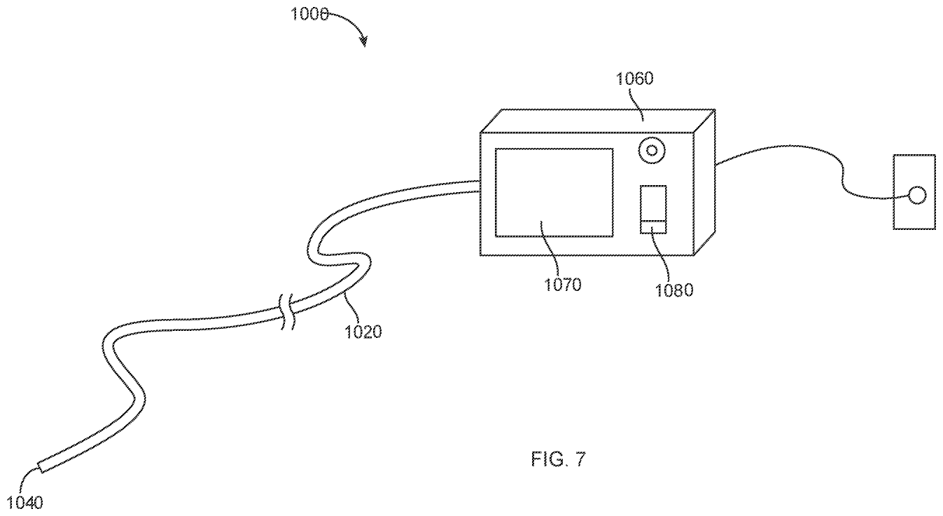

Referring now to FIG. 7, in one example, an elongate, medical device inspection scope 1000 includes an outer layer 1020 (or “outer housing”) and a distal end 1040. The proximal end of the scope 1000 is attached to a camera body/light source 1060, which may sometimes be referred to as a “box.” In some embodiments, the outer layer 1020 of the scope 1000 may have a specific diameter, sized to be able to fit within lumens of various endoscopes, catheters and/or other medical devices for inspection purposes. For example, in various embodiments, the outer layer 1020 may have an outer diameter of less than 2 millimeters, and in some embodiments less than 1 millimeter.

In one embodiment, the camera body/light source 1060 may include a display 1070 and one or more controllers 1080. The display 1070 may display an image of the inside of the medical device being examined, or it may display data related to the inside of the medical device and/or the scope 1000. In some embodiments, the camera body/light source 1060 may connect to a separate display monitor for displaying images captured by the scope 1000. The controllers 1080 may include an on/off power switch and any other switch, knob, controller or the like.

In some embodiments, the scope 1000 is configured to (1) emit illuminating light and capture still and/or video images of the inside of a medical device and (2) emit UV light (such as but not limited to UVC light) to clean/disinfect the inside/lumen of the medical device. Such an embodiment may be configured such that the user can switch back and forth between visible/illumination light and UVC light emission, and/or the user may in some cases emit both types of light simultaneously. In such embodiments, the controllers 1080 may include a function control switch, button, knob or the like, for switching back and forth between illumination mode, UVC light mode and in some examples a combination light mode. In one embodiment, the light selection controller 1080 may be a mechanical switch that allows the operator to toggle between visible/illumination light and UVC light. The switch may be rotary or leveler action and may be hand or foot operated. In another embodiment, the controller 1080 may be an electronically activated switch, such as an electronic button on the display 1070 or another screen, to switch between visible and UVC light. Embedded software, for example residing in the light source/box 1060, may be configured to control the UVC energy dwell time (on/off/pulsed—e.g., the amount of energy delivered over a specific time period) and intensity. In one embodiment, a connector (not shown) splices the two different inputs (i.e., the visible light and the UVC light) into one single output, while preventing the individual light energies from escaping and while withstanding the caustic nature of UVC light. In one embodiment, both wavelengths enter a housing through a standard connection point (not shown) and are then spliced into one exit point. In various examples, any suitable splice connector may be used.

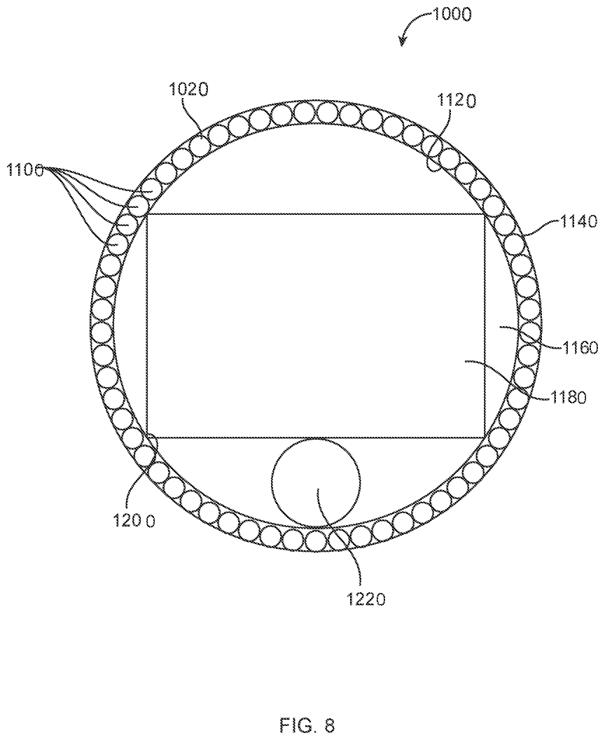

Referring to FIG. 8, the scope 1000 of FIG. 7 is illustrated in cross section. As seen in this figure, the scope 1000 includes the outer layer 1020 and an inner layer 1120, which together form a circumferential space 1140 between the two. Again, in some embodiments, the outer diameter of the outer layer 1020 may be less than 2 millimeters, or in some examples less than 1 millimeter. Multiple light fibers 1100 (or “illumination fibers”), which transmit light from the light source 1060 to the distal end 1040 of the scope 1000, may be positioned inside the circumferential space 1140 (as illustrated), inside the central lumen 1160, or both. Inside the inner layer 1120 is a central lumen 1160, which contains a camera module 1180 and an optional elongate stiffening member 1220. In this embodiment, the camera module 1180 has a rectangular cross-sectional shape. In an alternative embodiment, the corners 1200 of the camera module may be shaved, sanded or otherwise smoothed or rounded off.

As mentioned above, in some embodiments, the light fibers 1100 are configured to emit visible light for illumination purposes and ultraviolet light, such as UVC light, for disinfecting the inside of a medical device. As UVC light can be highly caustic, it is important to transmit the light down the length of the light fibers 1100 carefully, to prevent its unintended release. In some embodiments, each of the light fibers 1100 may include a silica core, a doped silica clad, a polyimide layer, and a buffer made of polyimide, silicone, acrylate, fluoropolymer or other suitable buffer material. These are merely examples, however, and in alternative embodiments other materials or combinations may be used. In some embodiments, all the light fibers 1100 may be configured to transmit visible light and UV light. In alternative embodiments, one set of fibers 1100 may be configured to transmit visible light, and another set of fibers 1100 may be configured to transmit UV light.

The stiffening member 1220 is an optional component, used in some embodiments to enhance/increase the rigidity of the scope 1000, to prevent over-bending or kinking. The stiffening member 1220 may have any suitable size, length, shape and material, according to various embodiments. In some embodiments, for example, the stiffening member 1220 may be a fiber, such as a glass or plastic fiber with a coating. In one embodiment, the stiffening member 1220 may be a laser fiber, which in the scope 100 is not used for transmitting light but only as a piece to add rigidity to the scope 1000.

In one embodiment, a method for making the medical device inspection scope 1000 may involve positioning the inner layer 1120 inside the outer layer 1020, then placing the multiple light fibers 1100 inside the circumferential space 1140. The camera module 1160 and laser fiber 1220 may then be placed in the inner lumen 1160 of the scope 1000. In one example, the method of making the scope 1000 may also include shaving, sanding or otherwise smoothing off corners 1200 of the camera module 1180.

A method for using the medical device inspection scope 1000 may involve inserting the distal end 1040 of the scope 1000 into a lumen of a medical device, such as an endoscope, catheter or any other suitable device. The scope 1000 is then advanced through the lumen, while the light fibers 1100 are used to illuminate the lumen, and the camera module 1160 is used to capture video and/or still images of the lumen. Some embodiments may include a processor for storing and/or interpreting data acquired by the camera module. For example, in some embodiments, the processor may be configured to identify irregularities or defects in the inside of an endoscope, catheter or other medical device.

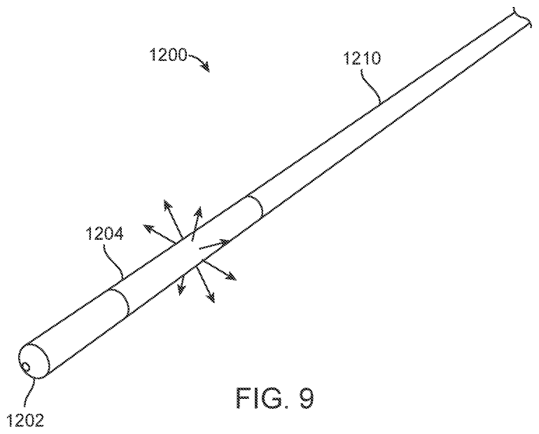

Referring now to FIG. 9, a distal portion of one embodiment of a UV laser fiber assembly 1200 for a medical device inspection and decontamination scope is illustrated. In this example, the UV laser fiber assembly 1200 includes a 1.3 mm diameter fiber 1210 with a distal end 1202 and a 10 mm long radial diffuser 1204 located just proximal of the distal end 1202. The diffuser may be reflective or non-reflective. UV light with a controlled power level is transmitted through the laser fiber 1210 from a source. With a known power level supplied by the light source, the illumination pattern emitting from the diffuser 1204 will be cylindrical in shape along the length of the diffuser 1204. The laser fiber assembly 1200 with the diffuser is inserted inside a lumen of a medical device having a diameter (d). The power density of the UV light transmitted to the inner surface of the lumen (P) is determined by the total output power of the diffuser (Pt) and the inner surface area of the lumen illuminated by the diffuser (A), defined as:

Power density=Total Power(mw)/Illumination area(cm²), or P=Pt/A.

In this example, the Power density is:

P=Pt/2π(d/2)(1 cm)mw/cm²

To control the energy and/or dosage delivered to the internal surface of the lumen requiring disinfection, the laser fiber assembly 1200 with the diffuser 1204 is drawn through the lumen of the medical device at a fixed speed, using the auto-feed function and artificial intelligence of the inspection/decontamination scope system. The diffuser 1204 will irradiate the inner surface of the lumen and deliver an energy level defined as Energy (mJ/cm²)=Power Density (mW/cm²)×T (seconds), where T is the total time the area of interest is illuminated by the UV diffuser 1204. In this example, the Energy delivered is:

E=Pt/2π(d/2)(1 cm)*T mJ/cm²

The above description is intended to be complete and accurate. It is meant to be a description of various embodiments, however, and is not intended to limit the scope of the invention. Various changes may be made to any of the embodiments described above, without departing from the scope of the invention described in the following claims. For example, features of one embodiment may be combined with a different embodiment, the order of steps in a given method may be changed, or the like.

We claim:

1. A fiber scope for inspecting an inner surface of a medical device, the fiber scope comprising:
a flexible, elongate shaft having a proximal end and a distal end;
a visualization member at or near the distal end of the shaft for visualizing the inner surface of the medical device; and
an ultraviolet light emitter at or near the distal end of the shaft for cleaning the inner surface, wherein the ultraviolet light emitter is configured to emit ultraviolet light in a uniform manner onto the inner surface.

2. The fiber scope of claim 1, wherein the ultraviolet light emitter is configured to emit UVC light.

3. The fiber scope of claim 1, wherein the ultraviolet light emitter comprises a laser fiber and a diffuser.

4. The fiber scope of claim 1, wherein the shaft has an outer diameter of no more than about 1.3 millimeters.

5. The fiber scope of claim 1, further comprising a connector attached to the proximal end of the shaft for connecting with a device housing an image processor.

6. The fiber scope of claim 1, further comprising a handle attached to the proximal end of the shaft and housing an image processor.

7. A method for inspecting and cleaning an inner surface of a medical device, the method comprising:
advancing a distal end of a flexible, elongate inspection scope into the medical device;
visualizing the inner surface of the medical device with a visualization member at or near the distal end of the inspection scope; and
emitting an ultraviolet light from an ultraviolet light emitter at or near the distal end of the inspection scope, to clean the inner surface, wherein the ultraviolet light emitter is configured to emit a uniform beam of ultraviolet light onto the inner surface.

8. The method of claim 7, wherein advancing the distal end is performed automatically by an auto-feed member coupled with the inspection scope.

9. The method of claim 7, further comprising identifying one or more contaminants on the inner surface of the medical device, using artificial intelligence in a processor coupled with the inspection scope.

10. The method of claim 7, wherein emitting the ultraviolet light comprises emitting UVC light.

11. The method of claim 7, wherein the ultraviolet light emitter comprises a laser fiber.

12. The method of claim 7, wherein the ultraviolet light emitter comprises a diffuser.

13. A method for disinfecting an endoscope or a catheter having a channel defined by an inner surface, the method comprising:
advancing a laser fiber assembly through the channel, the laser fiber assembly including an optical fiber having a radial light diffuser; and
transmitting an ultra-violet (UV) light from a light source through the optical fiber, the transmitted UV light being transmitted at a pre-determined power level, the transmission of the UV light causing emission of the UV light from the radial diffuser in a radial illumination pattern on the inner surface of the channel of the endoscope, the radial illumination pattern of UV light on the inner surface of the channel serving to irradiate and disinfect the inner surface.

14. The method of claim 13, wherein the radial light diffuser comprises an elongate radial light diffuser and wherein the radial illumination pattern is a cylindrical illumination pattern.

15. The method of claim 13, wherein the radial light diffuser is reflective.

16. The method of claim 13, wherein the radial light diffuser is non-reflective.

17. The method of claim 13, wherein the UV light comprises UVC light.

18. The method of claim 13, wherein advancing the laser fiber assembly comprises advancing the laser fiber assembly at a fixed speed.

19. The method of claim 13, wherein the inner surface of the channel is radiated for a predetermined amount of time.

20. The method of claim 13, wherein advancing the laser fiber assembly comprises auto-feeding the laser fiber assembly.
